(19) United States
(12) Patent Application Publication
(10) Pub. No.: US 2020/0077940 A1
(43) Pub. Date: Mar. 12, 2020

(54) VOICE ANALYSIS FOR DETERMINING THE CARDIAC HEALTH OF A SUBJECT

(71) Applicant: Cardiac Pacemakers, Inc., St. Paul, MN (US)

(72) Inventors: Kyle H. Srivastava, St. Paul, MN (US); Alexander J. Shrom, Shoreview, MN (US); Aaron P. Brooks, St. Paul, MN (US); Erika L. Williams, St. Paul, MN (US); Vinay Sircilla, Shoreview, MN (US)

(21) Appl. No.: 16/562,020

(22) Filed: Sep. 5, 2019

Related U.S. Application Data
(60) Provisional application No. 62/728,168, filed on Sep. 7, 2018.

(51) Int. Cl. A61B 5/00; A61B 5/02; G16H 40/63; G06N 20/00

(52) U.S. Cl. CPC A61B 5/4803; A61B 5/02028; A61B 5/7267; A61B 5/7275; G06N 20/00; A61B 5/742; A61B 5/0022; G16H 40/63; A61B 5/7278

(57) ABSTRACT

Embodiments for determining the cardiac health of a subject using voice analysis are disclosed. In an embodiment, a method comprises receiving a voice sample from the subject. The method further comprises determining one or more characteristics of the voice sample. And, the method further comprises determining the subject's cardiac health based on the one or more characteristics.

### CROSS REFERENCE TO RELATED APPLICATION

This application claims priority to Provisional Application No. 62/728,168, filed Sep. 7, 2018, all of which are herein incorporated by reference in their entirety.

### TECHNICAL FIELD

The present disclosure relates to determining a subject's cardiac health. More specifically, the present disclosure relates to system and methods for determining a subject's cardiac health using voice analysis.

### BACKGROUND

Subjects with heart conditions are susceptible to sudden, worsening of symptoms. The sudden, worsening symptoms can lead to emergency room visits, which can be expensive for subjects, hospitals and/or insurance companies.

### SUMMARY

Embodiments included herein facilitate determining the cardiac health of a subject using voice analysis. Example embodiments are as follows.

In an Example 1, a method for determining the cardiac health of a subject using voice analysis comprises: receiving a voice sample from the subject; determining one or more characteristics of the voice sample; and determining the subject's cardiac health based on the one or more characteristics.

In an Example 2, the method of Example 1, wherein determining the subject's cardiac health comprises determining the subject's cardiac health using machine learning techniques.

In an Example 3, the method of any one of Examples 1-2, further comprising storing a baseline voice sample and wherein determining the subject's cardiac health comprises comparing the one or more characteristics of the voice sample to one or more characteristics of the baseline voice sample.

In an Example 4, the method of Example 3, wherein the baseline voice sample is received from the subject.

In an Example 5, the method of any one of Examples 3-4, wherein the baseline voice sample is received from a group of individuals, wherein each individual of the group of individuals has at least one statistical characteristic that is similar to a statistical characteristic of the subject.

In an Example 6, the method of any one of Examples 1-5, wherein determining one or more characteristics of the voice sample comprises determining a frequency distribution of the voice sample and wherein determining the subject's cardiac health comprises determining the subject's cardiac health based on the frequency distribution of the voice sample.

In an Example 7, the method of any one of Examples 1-6, further comprising determining a cardiac health trend for the subject based on the subject's cardiac health determined at a first time and a second time, the second time occurring after the first time.

In an Example 8, the method of any one of Examples 1-7, further comprising stratifying the subject into a risk category based on the subject's cardiac health.

In an Example 9, the method of any one of Examples 1-8, further comprising receiving sensed data from a sensor associated with the subject and wherein determining the subject's cardiac health is based on the sensed data.

In an Example 10, the method of any one of Examples 1-9, further comprising receiving health data associated with the subject and wherein determining the subject's cardiac health comprises determining the subject's cardiac health based on the health data.

In an Example 11, the method of any one of Examples 1-10, wherein determining the subject's cardiac health comprises receiving whether the subject has experienced or is experiencing preserved ejection fraction or reduced ejection fraction and wherein determining the subject's cardiac health comprises determining the subject's cardiac health based on the whether the subject has experienced or is experiencing preserved ejection fraction or reduced ejection fraction.

In an Example 12, the method of any one of Examples 1-11, wherein receiving a voice sample from the subject comprises receiving a voice sample from the subject during a voice call in which the subject is participating.

In an Example 13, the method of any one of Examples 1-12, further comprising prompting the subject to elicit the voice sample.

In an Example 14, the method of any one of Examples 1-13, further comprising outputting to a display device a representation of the subject's cardiac health.

In an Example 15, a non-transitory computer readable medium having a computer program stored thereon for determining cardiac health of a subject using voice analysis, the computer program comprising instructions for causing one or more processors to: receive a voice sample from the subject; determine one or more characteristics of the voice sample; and determine the subject's cardiac health based on the one or more characteristics.

In an Example 16, a method for tracking cardiac health of a subject using voice analysis comprises receiving a voice sample from the subject; determining one or more characteristics of the voice sample; and determining the subject's cardiac health based on the one or more characteristics.

In an Example 17, the method of Example 16, wherein determining the subject's cardiac health comprises determining the subject's cardiac health using machine learning techniques.

In an Example 18, the method of Example 16, further comprising storing a baseline voice sample and wherein determining the subject's cardiac health comprises comparing the one or more characteristics of the voice sample to one or more characteristics of the baseline voice sample.

In an Example 19, the method of Example 18, wherein the baseline voice sample is received from the subject.

In an Example 20, the method of Example 18, wherein the baseline voice sample is received from a group of individuals, wherein each individual of the group of individuals has at least one statistical characteristic that is similar to a statistical characteristic of the subject.

In an Example 21, the method of Example 16, wherein determining one or more characteristics of the voice sample comprises determining a frequency distribution of the voice sample and wherein determining the subject's cardiac health comprises determining the subject's cardiac health based on the frequency distribution of the voice sample.

In an Example 22, the method of Example 16, further comprising determining a cardiac health trend for the subject based on the subject's cardiac health determined at a first time and a second time, the second time occurring after the first time.

In an Example 23, the method of Example 16, further comprising stratifying the subject into a risk category based on the subject's cardiac health.

In an Example 24, the method of Example 16, further comprising receiving sensed data from a sensor associated with the subject and wherein determining the subject's cardiac health is based on the sensed data.

In an Example 25, the method of Example 16, further comprising receiving health data associated with the subject and wherein determining the subject's cardiac health comprises determining the subject's cardiac health based on the health data.

In an Example 26, the method of Example 16, wherein determining the subject's cardiac health comprises receiving whether the subject has experienced or is experiencing preserved ejection fraction or reduced ejection fraction and wherein determining the subject's cardiac health comprises determining the subject's cardiac health based on the whether the subject has experienced or is experiencing preserved ejection fraction or reduced ejection fraction.

In an Example 27, the method of Example 16, wherein receiving a voice sample from the subject comprises receiving a voice sample from the subject during a voice call in which the subject is participating.

In an Example 28, the method of Example 16, further comprising prompting the subject to elicit the voice sample.

In an Example 29, the method of Example 16, further comprising outputting to a display device a representation of the subject's cardiac health.

In an Example 30, a non-transitory computer readable medium having a computer program stored thereon for determining cardiac health of a subject using voice analysis, the computer program comprising instructions for causing one or more processors to: receive a voice sample from the subject; determine one or more characteristics of the voice sample; and determine the subject's cardiac health based on the one or more characteristics.

In an Example 31, the non-transitory computer readable medium of Example 30, wherein to determine the subject's cardiac health, the computer program comprises instructions to determine the subject's cardiac health using machine learning techniques.

In an Example 32, the non-transitory computer readable medium of Example 30, the computer program comprising instructions to store a baseline voice sample and wherein to determine the subject's cardiac health, the computer program comprises instructions to compare the one or more characteristics of the voice sample to one or more characteristics of the baseline voice sample.

In an Example 33, the non-transitory computer readable medium of Example 32, wherein the baseline voice sample is received from the subject and/or a group of individuals, wherein each individual of the group of individuals has at least one statistical characteristic that is similar to a statistical characteristic of the subject.

In an Example 34, the non-transitory computer readable medium of Example 30, the computer program comprising instructions to determine a cardiac health trend for the subject based on the subject's cardiac health determined at a first time and a second time, the second time occurring after the first time.

In an Example 35, the non-transitory computer readable medium of Example 30, the computer program comprising instructions to stratify the subject into a risk category based on the subject's cardiac health.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a block diagram of system for determining the cardiac health of a subject using voice analysis, in accordance with embodiments of the present disclosure.

FIG. 2 is a block diagram depicting electronic devices and components included therein of the system of FIG. 1, in accordance with embodiments of the present disclosure.

FIG. 3 is a graph depicting a characteristic of a subject, in accordance with embodiments of the present disclosure.

FIG. 4 is a graph depicting a trend of a subject's cardiac health, in accordance with embodiments of the present disclosure.

FIG. 5 is a graph depicting a risk stratification of a subject's cardiac health, in accordance with embodiments of the present disclosure.

FIG. 6 is a flow diagram of a method for determining the cardiac health of a subject using voice analysis, in accordance with embodiments of the present disclosure.

While the disclosed embodiments are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

As stated above, sudden, worsening symptoms of heart conditions can lead to emergency room visits for subjects, which can be expensive for subjects, hospitals and/or insurance companies. The embodiments disclosed herein may facilitate identifying heart condition trends, which may prevent emergency room visits for subjects.

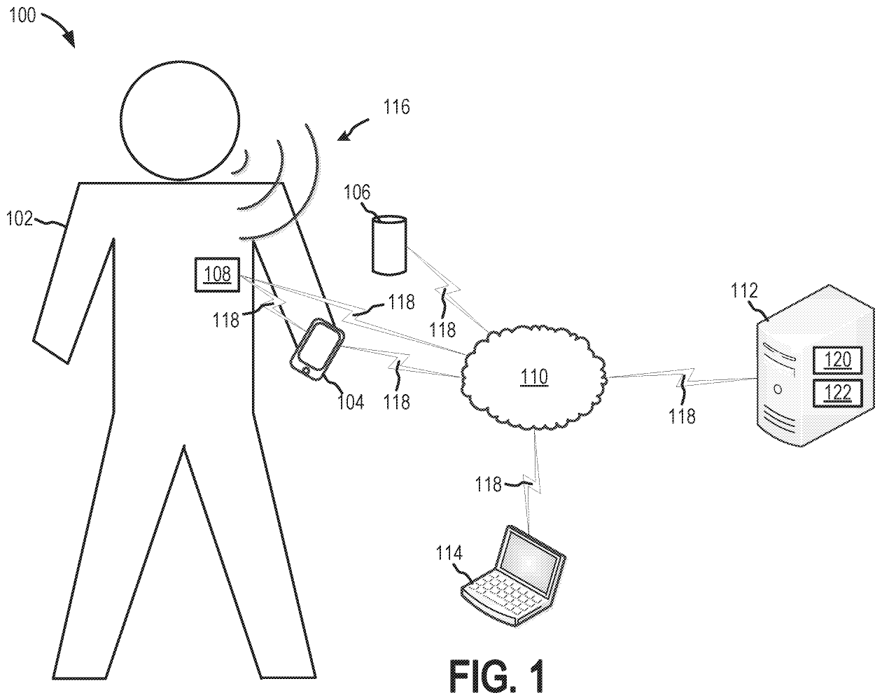

FIG. 1 is a block diagram of system 100 for determining the cardiac health of a subject 102 using voice analysis, in accordance with embodiments of the present disclosure. For example, when cardiac health begins to deteriorate, fluid in the lungs can accumulate. Accumulation of fluid in the lungs may also be referred to as pulmonary edema. When a subject 102 experiences pulmonary edema, characteristics of his/her voice may change. By receiving and analyzing a subject's 102 voice characteristics, the system 100 may determine the cardiac health of the subject 102.

While the present disclosure primarily discusses using voice analysis to determine a subject's 102 cardiac health, the embodiments disclosed herein may also be used to determine one or more of the following conditions which are also associated with pulmonary edema: acute respiratory distress syndrome, pneumonia, kidney failure, brain trauma, high altitudes, drug reactions, pulmonary embolisms, viral infections, eclampsia, smoke inhalation, and near drowning.

In embodiments, the system 100 may include a subject 102. The subject 102 may be a human, a dog, a pig, and/or any other animal having physiological parameters that can be recorded. For example, in embodiments, the subject 102 may be a human patient. In embodiments, the system 100 may also include a first exemplary electronic device 104, a second exemplary electronic device 106, a sensor device 108, a network 110, a server 112, and a third exemplary electronic device 114.

In embodiments, one or both of the electronic devices 104, 106 receive a voice sample 116 from the subject 102 when the subject 102 is speaking. And, one or both of the electronic devices 104, 106 that receive the voice sample 116 send data representing the voice sample 116 to the network 110 via a communication link 118 configured to communicate with the network 110.

The electronic devices 104, 106 include microphones for receiving the voice sample 116 and, in embodiments, memory for storing data representing the voice sample 116. One or both of the electronic devices 104, 106 are located near the subject 102 so one or both of the electronic devices 104, 106 can receive the voice sample 116. In embodiments, the electronic device 104 may be a wearable device (e.g., smartwatch, smart-glasses, and/or the like), a mobile device, such as a smartphone (e.g., an iPhone, an android phone, and/or the like), and the electronic device 106 may be a stationary device, such as a smart speaker (e.g., an Amazon Echo, Google Home, Sonos One, Apple HomePod, and/or the like), a smart TV, and/or the like. Alternatively, both the electronic devices 104, 106 may be mobile or both of the electronic devices 104, 106 may be stationary.

In embodiments, one or both of the electronic devices 104, 106 may be configured to remove ambient sound. Ambient sound may be any sound that is not the voice sample 116. For example, ambient sound may include sound emitted from the electronic devices 104, 106, sound from other sources in the adjacent environment, and/or the like. One or both of the electronic devices 104, 106 may distinguish ambient sound from the voice sample 116 by listening to sounds while not receiving the voice sample 116, characterizing those sounds (e.g., generating templates, models, waveforms, and/or the like that may be used to identify the sounds or similar sounds in subsequent samples), and remove those sounds from any received sound. As another example, one or both of the electronic devices 104, 106 may distinguish ambient sound from voice sample 116 by using voice recognition mechanisms to determine the voice of the subject 102 from other ambient sounds. Once the ambient sound is determined, the electronic devices 104, 106 may remove the ambient sound from recorded sound that includes the voice sample 116.

Additionally or alternatively, one or both of the electronic devices 104, 106 may include an altimeter. In these instances, one or both of the electronic devices 104, 106 may use a determined altitude to determine whether voice characteristics of the subject 102 are due to a change in cardiac health or a change in altitude.

In embodiments, a sensor device 108 may be associated with the subject 102. The sensor device 108 may be configured to send sensor data to the network 110 via a communication link 118 configured to communicate with the electronic device 104 and/or with the network 110. Sensor data from the sensor device 108, along with the voice sample 116, may facilitate determining the cardiac health of the subject 102.

The sensor device 108 may be configured to be positioned adjacent (e.g., on or near) the body of a subject 102. In embodiments, the sensor device 108 may provide one or more of the following functions with respect to a subject: sensing, data analysis, and/or therapy. For example, in embodiments, the sensor device 108 may be used to measure any number of a variety of physiological, device, subjective, and/or environmental parameters associated with the subject 102, using electrical, mechanical, and/or chemical means. The sensor device 108 may be configured to automatically gather data, gather data upon request (e.g., input provided by the subject, a clinician, another device, and/or the like), and/or any number of various combinations and/or modifications thereof. In embodiments, the sensor device 108 may include an electronics assembly configured to perform and/or otherwise facilitate any number of aspects of various functions.

The sensor device 108 may be configured to detect a variety of physiological signals that may be used in connection determining the subject's 102 cardiac health. For example, the sensor device 108 may include sensors or circuitry for detecting respiratory system signals, cardiac system signals, heart sounds, signals related to patient activity, and/or the like. Sensors and associated circuitry may be incorporated in connection with the sensor device 108 for detecting one or more body movement or body posture and/or position related signals. For example, accelerometers and/or GPS devices may be employed to detect patient activity, patient location, body orientation, and/or torso position. Environmental sensors may, for example, be configured to obtain information about the external environment (e.g., temperature, air quality, humidity, carbon monoxide level, oxygen level, barometric pressure, light intensity, sound, and/or the like) surrounding the subject 102. In embodiments, the sensor device 108 may be configured to measure any number of other parameters relating to or that might affect the human body, such as temperature (e.g., a thermometer), blood pressure (e.g., a sphygmomanometer), blood characteristics (e.g., glucose levels), body weight, physical strength, mental acuity, diet, heart characteristics, relative geographic position (e.g., a Global Positioning System (GPS)), and/or the like. Derived parameters may also be monitored using one or both of the electronic devices 104, 106.

According to embodiments, for example, the sensor device 108 may include one or more sensing electrodes configured to contact the body (e.g., the skin) of a subject 102 and to, in embodiments, obtain cardiac electrical signals. In embodiments, the sensor device 108 may include a motion sensor configured to generate an acceleration signal and/or acceleration data, which may include the acceleration signal, information derived from the acceleration signal, and/or the like. A “motion sensor,” as used herein, may be, or include, any type of accelerometer, gyroscope, inertial measurement unit (IMU), and/or any other type of sensor or combination of sensors configured to measure changes in acceleration, angular velocity, and/or the like.

The sensor device 108 may be configured to store data related to the physiological, device, environmental, and/or subjective parameters and/or transmit the data to any number of other devices in the system 100. In embodiments, the sensor device 108 may be configured to analyze data and/or act upon the analyzed data. For example, the sensor device 108 may be configured to modify therapy, perform additional monitoring, and/or provide alarm indications based on the analysis of the data.

In embodiments, the sensor device 108 may be configured to provide therapy. For example, the sensor device 108 may be configured to communicate with implanted stimulation devices, infusion devices, and/or the like, to facilitate delivery of therapy. The sensor device 108 may be, include, or be included in a medical device (external and/or implanted) that may be configured to deliver therapy. Therapy may be provided automatically and/or upon request (e.g., an input by the subject 102, a clinician, another device or process, and/or the like). The sensor device 108 may be programmable in that various characteristics of its sensing, therapy (e.g., duration and interval), and/or communication may be altered by communication between the sensor device 108 and other components of the system 100.

According to embodiments, the sensor device 108 may include any type of medical device, any number of different components of an implantable or external medical system, a mobile device, a mobile device accessory, and/or the like. In embodiments, the sensor device 108 may include a mobile device, a mobile device accessory such as, for example, a device having an electrocardiogram (ECG) module, a programmer, a server, and/or the like. In embodiments, the sensor device 108 may include a medical device. That is, for example, the sensor device 108 may include a control device, a monitoring device, a pacemaker, an implantable cardioverter defibrillator (ICD), a cardiac resynchronization therapy (CRT) device and/or the like, and may be an implantable medical device known in the art or later developed, for providing therapy and/or diagnostic data about the subject 102. In various embodiments, the sensor device 108 may include both defibrillation and pacing/CRT capabilities (e.g., a CRT-D device). In embodiments, the sensor device 108 may be implanted subcutaneously within an implantation location or pocket in the patient's chest or abdomen and may be configured to monitor (e.g., sense and/or record) physiological parameters associated with the subject's 102 heart. In embodiments, the sensor device 108 may be an implantable cardiac monitor (ICM) (e.g., an implantable diagnostic monitor (IDM), an implantable loop recorder (ILR), etc.) configured to record physiological parameters such as, for example, one or more cardiac electrical signals, heart sounds, heart rate, blood pressure measurements, oxygen saturations, and/or the like.

In various embodiments, the sensor device 108 may be a device that is configured to be portable with the subject 102, e.g., by being integrated into a vest, belt, harness, sticker; placed into a pocket, a purse, or a backpack; carried in the subject's hand; and/or the like, or otherwise operably (and/or physically) coupled to the subject 102. The sensor device 108 may be configured to monitor (e.g., sense and/or record) physiological parameters associated with the subject 102 and/or provide therapy to the subject 102. For example, the sensor device 108 may be, or include, a wearable cardiac defibrillator (WCD) such as a vest that includes one or more defibrillation electrodes. In embodiments, the sensor device 108 may include any number of different therapy components such as, for example, a defibrillation component, a drug delivery component, a neurostimulation component, a neuromodulation component, a temperature regulation component, and/or the like. In embodiments, the sensor device 108 may include limited functionality, e.g., defibrillation shock delivery and communication capabilities, with arrhythmia detection, classification and/or therapy command/control being performed by a separate device.

The network 110 may be any number of different types of communication networks such as, for example, a bus network, a short messaging service (SMS), a local area network (LAN), a wireless LAN (WLAN), a wide area network (WAN), the Internet, a P2P network, custom-designed communication or messaging protocols, and/or the like. Additionally or alternatively, the network 110 may include a combination of multiple networks, which may be wired and/or wireless.

The communication links 118 may be, or include, a wired link (e.g., a link accomplished via a physical connection) and/or a wireless communication link such as, for example, a short-range radio link, such as Bluetooth, IEEE 802.11, near-field communication (NFC), WiFi, a proprietary wireless protocol, and/or the like. The term “communication link” may refer to an ability to communicate some type of information in at least one direction between at least two devices, and should not be understood to be limited to a direct, persistent, or otherwise limited communication channel. That is, according to embodiments, the communication link 118 may be a persistent communication link, an intermittent communication link, an ad-hoc communication link, and/or the like. The communication link 118 may refer to direct communications between the components of the system 100, and/or indirect communications that travel between the components of the system 100 via at least one other device (e.g., a repeater, router, hub, and/or the like). The communication link 118 may facilitate uni-directional and/or bi-directional communication between the components of the system 100. Data and/or control signals may be transmitted between the components of the system 100 to coordinate the functions of the components of the system 100. In embodiments, subject data may be downloaded from one or more of the electronic devices 104, 106, the sensor 108 and/or other components of the system 100 periodically or on command. A clinician and/or the subject 102 may communicate with the components of the system 100, for example, to acquire subject data or to initiate, terminate and/or modify recording and/or therapy.

In embodiments, the network 110 sends data representing the voice sample 116 to the server 112 via a communication link 118. The server 112 analyzes the data representing the voice sample 116 to determine the cardiac health of the subject 102. Additionally or alternatively to the server 112 analyzing the data representing the voice sample 116 to determine the cardiac health of the subject 102, one or both of the electronic devices 104 may analyze the data representing the voice sample 116 to determine the cardiac health of the subject 102.

In embodiments, the server 112 may include, for example, a processor 120 and memory 122. The processor 120 may include, for example, a processing unit, a pulse generator, a controller, a microcontroller, and/or the like. The processor 120 may be any arrangement of electronic circuits, electronic components, processors, program components and/or the like configured to store and/or execute programming instructions, to direct the operation of the other functional components of the server 112. For example, the processor 120 may control the storage of data representing the voice sample 116 on memory 122 and/or determine the cardiac health of the subject 102 based on the data representing the voice sample 116.

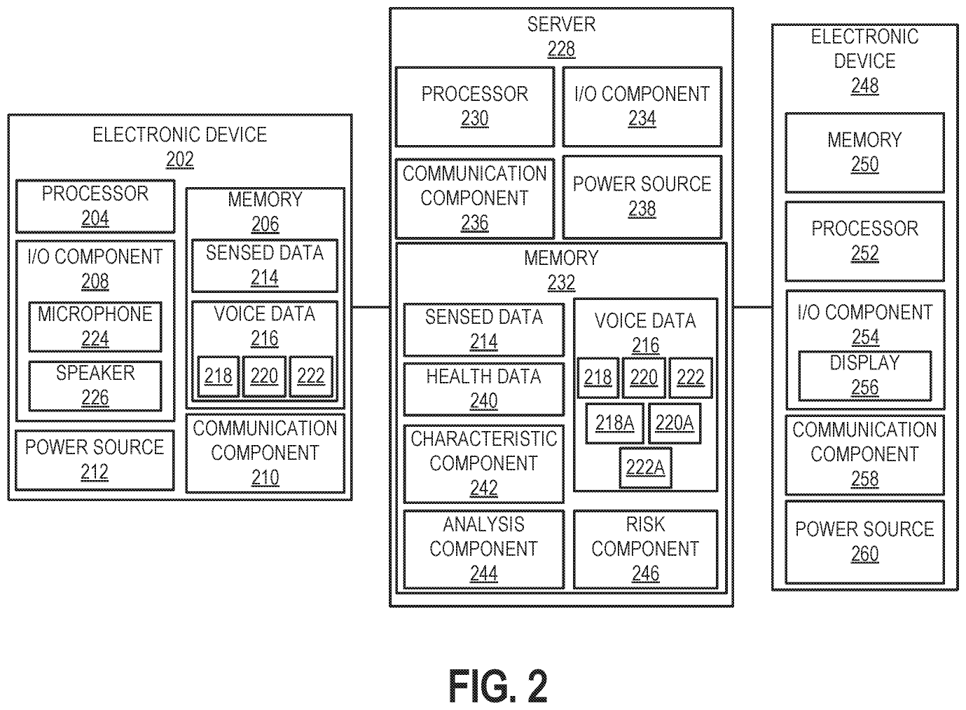

In embodiments, the processor 120 may represent a single processor 106 or multiple processors 106, and the single processor 120 and/or multiple processors 120 may each include one or more processing circuits. The processor 120 may include one or more processing circuits, which may include hardware, firmware, and/or software. In embodiments, different processing circuits of the processor 120 may perform different functions. For example, the processor 120 may include a first processing circuit configured to store the data representing the voice sample 116, a second processing circuit configured to classify the voice sample 116, and a third processing circuit configured to determine the cardiac health of the subject 102 based on the voice sample 116, as discussed in further detail below in relation to FIGS. 2-6.

In embodiments, the processor 120 may be a programmable micro-controller or microprocessor, and may include one or more programmable logic devices (PLDs) or application specific integrated circuits (ASICs). In some implementations, the processor 120 may include memory as well. The processor 120 may include digital-to-analog (D/A) converters, analog-to-digital (ND) converters, timers, counters, filters, switches, and/or the like. The processor 106 may execute instructions and perform desired tasks as specified by the instructions.

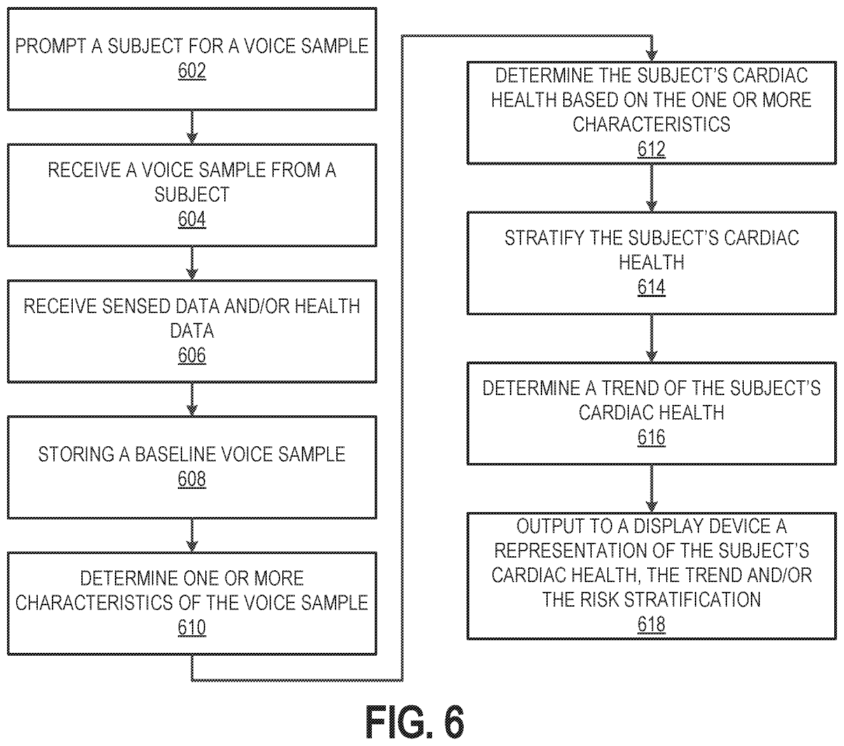

As stated above, the processor 120 may also be configured to store information in the memory 122 (e.g., data representing the voice sample 116) and/or access information from the memory 122. The memory 122 may include volatile and/or non-volatile memory, and may store instructions that, when executed by the processor 106 cause programming components, for example, the components depicted in FIG. 2, and/or methods (e.g., algorithms) to be performed, for example, the method 600 depicted in FIG. 6.

In embodiments, the results of the cardiac health analysis may be transmitted from the server 112 to one or more of the electronic devices 104, 106, 114 via the network 110 and one or more communication links 118. In embodiments where one or more of the electronic devices 104, 106 determines the cardiac health of the subject 102 based on the voice sample 116, the one or more of the electronic devices 104, 106 may transmit the results to the server 112 and/or the electronic device 114 via the network 110 and one or more communication links 118.

In embodiments, the electronic device 114 is accessible by a clinician to review the determined cardiac health of the subject 102. The review of the cardiac health by the clinician may result in a report that can be transmitted to one or both of the electronic devices 104, 106 via the network 110 so the report can be received by the subject 102. Additionally or alternatively, the report can be transmitted to the server 112 for storage and/or analysis. In embodiments, the clinician may also send medical advice (e.g., prescriptions, dietary restrictions, behavioral changes and/or the like) to the subject 102 upon reviewing the cardiac health of the subject 102.

The illustrative system 100 shown in FIG. 1 is not intended to suggest any limitation as to the scope of use or functionality of embodiments of the present disclosure. The illustrative system 100 should not be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the subject matter disclosed herein.

Referring to FIG. 2, a block diagram depicting exemplary components that may be included in the system 100 of FIG. 1 is illustrated. The illustrated embodiment includes an electronic device 202. The electronic device 202 may be used as the electronic device 104 and/or the electronic device 106 of the system 100 depicted in FIG. 1.

In embodiments, the electronic device 202 includes a processor 204, memory 206, an I/O component 206, a communication component 208, and a power source 210. Any number of the different illustrated components may represent one or more of said components. The processor 204 may include, for example, one or more processing units, one or more pulse generators, one or more controllers, one or more microcontrollers, and/or the like. The processor 204 may be any arrangement of electronic circuits, electronic components, processors, program components and/or the like configured to store and/or execute programming instructions, to direct the operation of the other functional components of the electronic device 202, to perform processing on any sounds sensed by the I/O component 208, perform processing on any sensed data from a sensor (e.g., the sensor 108 of FIG. 1), instruct the communication component 210 to transmit data and/or receive data, and may be implemented, for example, in the form of any combination of hardware, software, and/or firmware.

In embodiments, the processor 204 may be, include, or be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more central processing units (CPUs), software, hardware, firmware, or any combination of these and/or other components. According to embodiments, the processor 204 may include a processing unit configured to communicate with memory 206 to execute computer-executable instructions stored in the memory 206. As indicated above, although the processor 204 is referred to herein in the singular, the processor 204 may be implemented in multiple instances, distributed across multiple sensing devices, instantiated within multiple virtual machines, and/or the like.

The processor 204 may also be configured to store information in the memory 206 and/or access information from the memory 206. For example, the processor 204 may be configured to store data obtained by a sensor (e.g., the sensor 108) as sensed data 214 in memory 206. The sensed data 214 may include any of the data sensed by the sensor 108 as discussed in relation to FIG. 1. For example, sensed data 214 may include one or more locations, physiological parameters, device parameters, and/or environmental parameters. Physiological parameters may include, for example, cardiac electrical signals, respiratory signals, heart sounds, chemical parameters, body temperature, activity parameters, and/or the like. Device parameters may include any number of different parameters associated with a state of the sensor 108 and/or any other device (e.g., the electronic device 202) and may include, for example, battery life, end-of-life indicators, processing metrics, and/or the like. Environmental parameters may include particulates, ultraviolet light, volatile organic compounds, and/or the like in the environment. The physiological parameters may include respiratory parameters (e.g., rate, depth, rhythm), motion parameters, (e.g., walking, running, falling, gait, gait rhythm), facial expressions, swelling, heart sounds, sweat, sweat composition (e.g., ammonia, pH, potassium, sodium, chloride), exhaled air composition, Electrocardiography (ECG) parameters, electroencephalogram (EEG) parameters, Electromyography (EMG) parameters, and/or the like. Additionally or alternatively, location data indicative of the location of the sensor 108 may be saved as sensed data 214. In embodiments, the sensed data 214 may be used to determine the cardiac health of a subject (e.g., the subject 102 of FIG. 1) as discussed in more detail below.

According to embodiments, the processor 204 may be configured to store voice data obtained by the I/O component 208 as voice data 216. As stated above, the voice data 216 may be used to determine the cardiac health of the subject, as explained in more detail below. In embodiments, the voice data 216 may include one or more different types of voice data. For example, the voice data 216 may include voice data 218 received from the subject 102 at a plurality of times. For example, the voice data 216 may include voice data 218 received from the subject 102 at a first time and voice data 220 received from the subject 102 at a second time such that the second time occurs after the first time. Additionally or alternatively, the voice data 216 may include voice data 222 received from a group of subjects. In embodiments, the group of subjects may or may not include the subject 102. In embodiments, the group of subjects may have one or more characteristics that are the same or similar to the subject. Example characteristics include, but are not limited to, age, sex, blood pressure (systolic and/or diastolic), cholesterol (total, LDL and/or HDL), weight, smoking status, medication adherence (using, e.g., a connected pillbox), patient reported information (e.g., diet, exercise, mood, sleep duration, quality of sleep, and/or the like), a health assessment, creatinine, hemoglobin, triglycerides, body-mass index, medical history (e.g., treated hypertension, treated hyperlipidemia, chronic kidney disease, peripheral vascular disease, transient ischemic attack, cerebrovascular accident, edema, diabetic history, atherosclerotic cardiovascular disease history and/or risk score, and/or the like), family medical history and/or the like. In embodiments, the voice data 218 and/or the voice data 222 may be referred to herein as baseline voice data determined from a baseline voice sample.

In embodiments, the memory 206 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory stores computer-executable instructions for causing the processor to implement aspects of embodiments of system components discussed herein and/or to perform aspects of embodiments of methods and procedures discussed herein.

Computer-executable instructions stored on memory 206 may include, for example, computer code, machine-useable instructions, and the like such as, for example, program components capable of being executed by one or more processors associated with the computing device. Program components may be programmed using any number of different programming environments, including various languages, development kits, frameworks, and/or the like. Some or all of the functionality contemplated herein may also, or alternatively, be implemented in hardware and/or firmware.

The I/O component 208 may include and/or be coupled to a microphone 224 for receiving a voice sample (e.g., the voice sample 116 of FIG. 1) from the subject (e.g., the subject 102). In embodiments, the voice sample may be received by the microphone 224 from the subject when the subject is on a voice call using the electronic device 202. Additionally or alternatively, the I/O component 208 may also include a speaker 226, which, in response to instructions stored on memory 206 being executed by the processor 204, may provide an impetus to the subject 102 in order to elicit a response and, therefore, a voice sample 116 from the subject 102. The impetus may be an indication to speak (e.g., a beep), a question, and/or the like. Additionally or alternatively, the I/O component 208 may provide a visual impetus to speak in order to elicit a voice sample from the subject 102. In embodiments, the impetus provide by the speaker 226 may be configured to elicit different types of responses. For example, the impetus may be a request that the subject: speak predefined words, describe a positive emotional experience, describe a negative emotional experience, describe his/her daily activities, and/or the like. While the discussion herein relates to receiving a voice sample, the voice sample may comprise multiple voice samples.

Once the impetus is provided, the speaker 226 can receive a voice sample (e.g., the voice sample 116) in response to the impetus. In embodiments, the processor 204 may be configured to process the voice sample and determine whether the voice sample satisfies one or more criteria. The one or more criteria may facilitate determining whether the voice sample is sufficient to be used to determine the cardiac health of the subject. In embodiments, the one or more criteria may be characteristics of the voice sample (e.g., the length of the sample, the amplitude (i.e., loudness) of the sample, and/or the like). In embodiments, if the voice sample does not satisfy the one or more criteria, the electronic device 202 via the speaker 226 may provide a subsequent impetus to the subject 102 in order to elicit another voice sample. In embodiments, the subsequent impetus may also be provided with an explanation as to why another voice sample is being elicited.

Additionally or alternatively, the I/O component 208 may include a user interface configured to present information to a user or receive an indication from a user. For example, the I/O component 208 may include and/or be coupled to a display device, a printing device, a light emitting diode (LED), and/or the like, and/or an input component such as, for example, a button, a joystick, a satellite dish, a scanner, a printer, a wireless device, a keyboard, a pen, a voice input device, a touch input device, a touch-screen device, an interactive display device, a mouse, and/or the like. In embodiments, the I/O component 208 may be used to present and/or provide an indication of any of the data sensed and/or produced by the electronic device 202 and/or any other components depicted in FIGS. 1 and 2.

The communication component 210 may be configured to communicate (i.e., send and/or receive signals) with the electronic device 202 and/or other devices such as those included in FIGS. 1 and 2. For example, the communication component 210 may be configured to receive sensed data 214 from the sensor 108 and/or send sensed data 214 and/or voice data 216 to the server 228. The communication component 210 may include, for example, circuits, program components, and one or more transmitters and/or receivers for communicating wirelessly with one or more other devices such as, for example, the server 228. According to various embodiments, the communication component 210 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 210 may include any combination of hardware, software, and/or firmware configured to facilitate establishing, maintaining, and using any number of communication links.

The power source 212 provides electrical power to the other operative components (e.g., the processor 204, the memory 206, the I/O component 208, and/or the communication component 210, and may be any type of power source suitable for providing the desired performance and/or longevity requirements of the electronic device 202. In various embodiments, the power source 212 may include one or more batteries, which may be rechargeable (e.g., using an external energy source). The power source 212 may include one or more capacitors, energy conversion mechanisms, and/or the like. Additionally or alternatively, the power source 212 may harvest energy from a subject (e.g., the subject 102) (e.g. motion, heat, biochemical) and/or from the environment (e.g. electromagnetic). Additionally or alternatively, the power source 212 may harvest energy from an energy source connected to the body, for example, a shoe may receive energy from impact and send the received energy to a power source 212 of the electronic device 202.

The illustrated embodiment of FIG. 2 also includes a server 228. In embodiments, the server 228 may include a processor 230, memory 232, an I/O component 234, a communication component 236, and/or a power source 238.

The processor 230 may include, for example, one or more processing units, one or more pulse generators, one or more controllers, one or more microcontrollers, and/or the like. The processor 230 may be any arrangement of electronic circuits, electronic components, processors, program components and/or the like configured to store and/or execute programming instructions, to direct the operation of the other functional components of the server 228 and may be implemented, for example, in the form of any combination of hardware, software, and/or firmware.

In embodiments, the processor 228 may be, include, or be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more central processing units (CPUs), software, hardware, firmware, or any combination of these and/or other components. According to embodiments, the processor 228 may include a processing unit configured to communicate with memory 232 to execute computer-executable instructions stored in the memory 232. As indicated above, although the processor 230 is referred to herein in the singular, the processor 230 may be implemented in multiple instances, distributed across multiple sensing devices, instantiated within multiple virtual machines, and/or the like.

The processor 230 may be configured to store information in the memory 232 and/or access information from the memory 232. For example, the processor 230 may be configured to store sensed data 214 received from the electronic device 202. As another example, the processor 230 may be configured to store voice data received from the electronic device 202, which may include voice data 218 received from the subject 102 at a first time, voice data 220 received from the subject 102 at a second time where the second time occurs after the first time, voice data 222 received from a group of subjects, where the group of subjects may or may not include the subject 102. The sensed data 214 and/or the voice data 216 may be used to determine the cardiac health of the subject, as explained in more detail below. In addition, while the embodiments discuss storing voice data 216 received from the subject at a first time and a second time, the voice data 216 may be received at a third time, fourth time, etc. where the third time occurs after the second time, the fourth time occurs after the third time, etc. Furthermore, ambient noise may be removed from the voice data 218, the voice data 220, and/or the voice data 222.

In embodiments, the memory 232 may include health data 240, a characteristic component 242, an analysis component 244, and/or a risk component 246, which include respective instructions that can be executed by the processor 230. While the health data 240, characteristic component 242, analysis component 244, and risk component 246 are depicted as being included in the memory 232, additionally or alternatively, the health data 240, the characteristic component 242, analysis component 244, and the risk component 246 may be included in the memory 206 and executed by the processor 204. Additionally or alternatively, the health data 240, the characteristic component 242, analysis component 244, and the risk component 246 may be included in the memory 206 and executed by the processor 204 may be included in memory 250 of the electronic device 248.

In embodiments, the health data 240 may be used to supplement the voice sample and/or the sensor data 214 to determine the subject's cardiac health (and/or one of the other conditions discussed above, e.g., pulmonary edema, acute respiratory distress syndrome, pneumonia, kidney failure, brain trauma, high altitudes, drug reactions, pulmonary embolisms, viral infections, eclampsia, smoke inhalation, and near drowning), as discussed in more detail below. The health data 240 may be input into the electronic device 202 and transferred to the server 228, input into the server 228, input into the electronic device 248 and transferred to the server 228, received from medical records on the subject and/or the like. The health data 240 may include, for example, age, sex, blood pressure (systolic and/or diastolic), cholesterol (total, LDL and/or HDL), weight, smoking status, medication adherence (using, e.g., a connected pillbox), patient reported information (e.g., diet, exercise, mood, sleep duration, quality of sleep, and/or the like), a health assessment, creatinine, hemoglobin, triglycerides, body-mass index, medical history (e.g., treated hypertension, treated hyperlipidemia, chronic kidney disease, peripheral vascular disease, transient ischemic attack, cerebrovascular accident, edema, diabetic history, atherosclerotic cardiovascular disease history and/or risk score, and/or the like), family medical history and/or the like.

The characteristic component 242 is configured to determine one or more characteristics of the voice data 216. In embodiments, determining one or more characteristics of the voice data 216 may include, in the event the follow voice data 216 is available: determining one or more characteristics of the voice data 218 received from the subject at a first time, determining one or more characteristics of the voice data 220 received from the subject at a second time, and/or determining one or more characteristics of the voice data 222 received from a group of subjects. The one or more characteristics of the voice data 218 received from the subject at a first time may be saved in memory 232 as voice characteristic data 218A; the one or more characteristics of the voice data 220 received from the subject at a second time may be saved in memory 232 as voice characteristic data 220A; and, the one or more characteristics of the voice data 222 received from a group of subjects may be saved in memory 232 as voice characteristic data 222A.

An example characteristic 218A, 220A, 222A that the characteristic component 242 may determine from the voice data 216 is the frequency as a function of time of the voice data 216. As another example characteristic 218A, 220A, 222A, the characteristic component 242 may determine the amplitude as a function of frequency. Other example characteristics 218A, 220A, 222A include, but are not limited to, phonatory regularity, fundamental frequency, fundamental frequency median, fundamental frequency standard deviation, cepstral peak prominence, low-high spectral ratio, jitter in speech, durations of speech breath groups, pausing in speech, creak in speech, total breath group duration, mean phenomes per phrase, max phonemes per phrase, phenomes standard deviation per phrase, and/or the like. Other exemplary characteristics 218A, 220A, 222A are described in, for example, “Acoustic speech analysis of patients with decompensated heart failure: A pilot study,” authored by Murton, Olivia M, Hillman, Robert E., Mehta, Daryush D., Semigran, Marc, Daher, Maureen, Cunningham, Thomas, Verkouw, Karla, Tabtabai, Sara, Steiner, Johannes, Dec, G. William, and Ausiello, Dennis, available at https://doi.org/10.1121/1.5007092, and “Voice Signal Characteristics Are Independently Associated With Coronary Artery Disease,” authored by Maor, Elad, Sara, Jaskanwal D, Orbelo, Diana M, Lerman, Lilach O., Levanon, Yoram, and Lerman, Amir, available at https://www.mayoclinicproceedings.org/article/S0025-6196(18)30030-2/fulltext, the entireties of both of which are hereby incorporated herein by reference for all purposes.

In embodiments, the analysis component 242 is configured to determine the subject's cardiac health from the one or more characteristics 218A, 220A, 222A. For example, in embodiments where the characteristics 218A and/or the characteristics 220A are determined by the characteristic component 242, the analysis component 242 may determine correlations between the one or more characteristics 218A, 220A and cardiac health. To do so, the analysis component 242 may: (i) receive one or more characteristics extracted from voice samples of one or more subjects (the one or more characteristics may be extracted by the characteristic component 242), (ii) receive cardiac health indicators of the sample of subjects, and (iii) determine correlations therebetween using machine learning techniques. And, based on the correlations between the one or more characteristics and the cardiac health of the one or more subjects, the analysis component 242 may determine the cardiac health of the subject 102 based on the characteristics 218A and/or the characteristics 220A. Example learning techniques include, but are not limited to, one or more of the following techniques: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), and any other suitable learning style.

In embodiments, the analysis component 242 may also incorporate the sensed data 214 and/or the health data 240 into determining correlations between the one or more characteristics 218A and/or the one or more characteristics 220A and cardiac health. For example, an increase (or decrease) of a first sensed data of the sensed data 214 in addition to an increase (or decrease) of a first characteristic of the characteristics 218A and/or the characteristics 220A may indicate an increase (or decrease) in cardiac health whereas an increase (or decrease) of the first sensed data by itself or the first characteristic by itself may be indeterminate as to whether the subject's cardiac health is increasing, decreasing, or stable. As another example, an increase (or decrease) of a first health data of the health data 240 in addition to an increase (or decrease) of a first characteristic of the characteristics 218A and/or the characteristics 220A may indicate an increase (or decrease) in cardiac health whereas an increase (or decrease) of the first health data by itself or the first characteristic by itself may be indeterminate as to whether the subject's cardiac health is increasing, decreasing, or stable. As even another example, an increase (or decrease) of a first sensed data of the sensed data 214, in addition to an increase (or decrease) of a first health data of the health data 240, and in addition to an increase (or decrease) of a first characteristic of the characteristics 218A and/or the characteristics 220A may indicate an increase (or decrease) in cardiac health whereas an increase (or decrease) of two of the three (i.e., the first sensed data, the first health data and the first characteristic) may be indeterminate as to whether the subject's cardiac health is increasing, decreasing, or stable.

As another example, in embodiments where the characteristics 218A and the characteristics 220A are determined by the characteristic component 242, the analysis component 242 may compare one or more of the characteristics 218A with one or more of the characteristics 220A. Based on the comparison, the analysis component 242 may determine the subject's cardiac health. For example, if a first characteristic of the characteristics 220A increases (or decreases) in comparison to the first characteristic of the characteristics 218A, and an increase (or decrease) in the first characteristic is correlated to an increase (or decrease) in cardiac health, the analysis component 242 may determine the subject's cardiac health is increasing (or decreasing). Additionally or alternatively, because the characteristics 218A are determined at a first time and the characteristics 220A are determined at a second time, where the second time is after the first time, the analysis component 242 may plot of trend of the subject's cardiac health. That is, the analysis component 242 may plot the subject's cardiac health at the first time and the subject's cardiac health at the second time (and a third time, fourth time, etc.).

In embodiments where the characteristics 218A and the characteristics 222A are determined by the characteristic component 242, the analysis component 242 may compare one or more of the characteristics 218A with one or more of the characteristics 222A. Based on the comparison, the analysis component 242 may determine the subject's cardiac health. For example, if a first characteristic of the characteristics 218A is greater (or less) than the first characteristic of the characteristics 222A, and being greater (or less) than the first characteristic of the characteristics 222A is correlated to better (or worse) cardiac health, the analysis component 242 may determine the subject's cardiac health is better (or worse) than the cardiac health of the group of subjects from which the characteristics 222A are determined.

In embodiments, the risk component 246 may determine the risk associated with the subject's cardiac health determined by the analysis component 244. For example, the risk component 246, based on the determined cardiac health of the subject, may determine the likelihood the subject has experienced, is experiencing or may experience one or more cardiac events (e.g., preserved ejection fraction, reduced ejection fraction) and/or the severity of the one or more cardiac events. Additionally or alternatively, based on the determined cardiac health of the subject, the risk component 246 may determine the benefits and/or detriments to: a lifestyle change, a surgical procedure, starting (or ceasing) a medication and/or the like. Additionally or alternatively, based on the determined cardiac health of the subject, the risk component 246 may assign a score to the subject's cardiac health, which may correlate to one or more indicators and/or scores (e.g., the Cardiovascular Health score).

By determining the subject's cardiac health, a risk associated with the subject's cardiac health and/or a trend of the subject's cardiac health, intervention to increase the subject's cardiac health may be taken prior to the subject having to visit an emergency room, which may save money and/or resources spent by or on the subject.

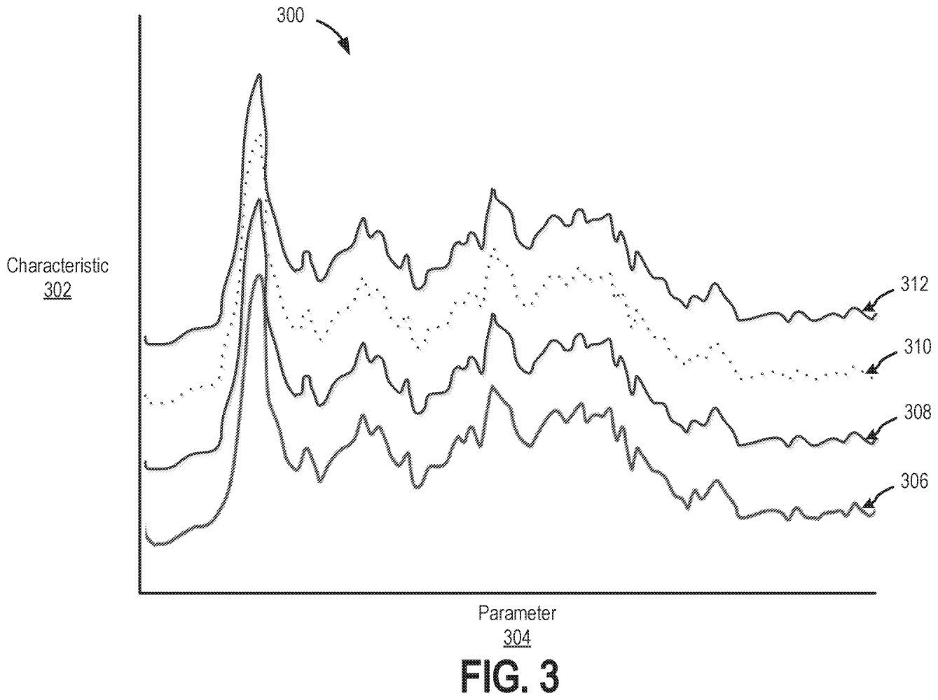

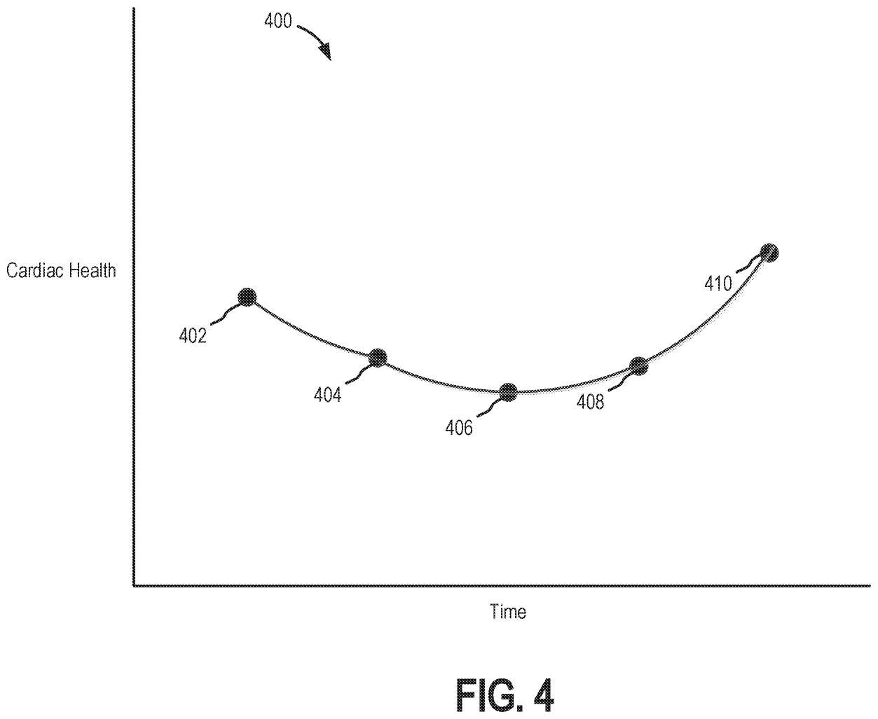

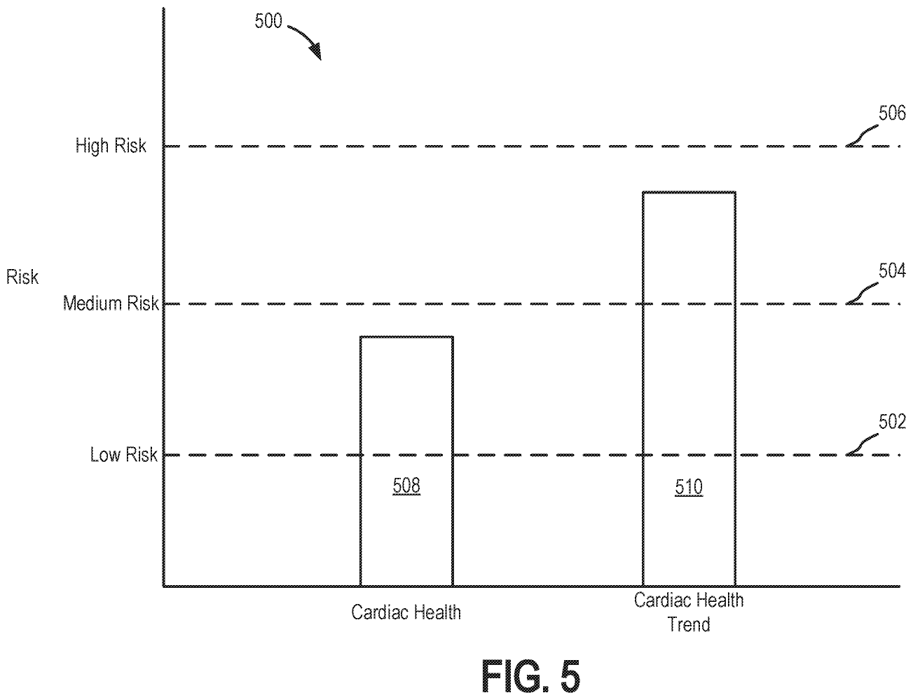

In embodiments, a representation of one or more of the characteristics 218A, 220A, 222A may be output to the I/O component's 208 display device via the communication component 210 and/or the I/O component's 254 display device 256 (of the electronic device 248) via the communication component 258. Additionally or alternatively, a representation of the subject's cardiac health and/or a representation of a trend of the subject's cardiac health may be output to the I/O component's 208 display device via the communication component 210 and/or the I/O component's 254 display device 256 via the communication component 258. An example representation of a characteristic of the characteristics 218A, 220A, 222A and an example representation of the determination of the subject's cardiac health are depicted in FIG. 3. An example representation of a trend of the subject's cardiac health is depicted in FIG. 4. Additionally or alternatively, a representation of the risk associated with the subject's cardiac health may be output to the I/O component's 208 display device via the communication component 210 and/or the I/O component's 254 display device 256 via the communication component 258. An example representation of the risk associated the subject's cardiac health is depicted in FIG. 5.

In embodiments, the memory 232 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory stores computer-executable instructions for causing the processor to implement aspects of embodiments of system components discussed herein and/or to perform aspects of embodiments of methods and procedures discussed herein.

Computer-executable instructions stored on memory 232 may include, for example, computer code, machine-useable instructions, and the like such as, for example, program components capable of being executed by one or more processors associated with the computing device. Program components may be programmed using any number of different programming environments, including various languages, development kits, frameworks, and/or the like. Some or all of the functionality contemplated herein may also, or alternatively, be implemented in hardware and/or firmware.

In embodiments, the I/O component 234 may include a user interface configured to present information to a user or receive indication from a user. For example, the I/O component 242 may include and/or be coupled to a display device, a printing device, a speaker, a light emitting diode (LED), and/or the like, and/or an input component such as, for example, a button, a joystick, a microphone, a satellite dish, a scanner, a printer, a wireless device, a keyboard, a pen, a voice input device, a touch input device, a touch-screen device, an interactive display device, a mouse, and/or the like. In embodiments, the I/O component 234 may be used to present and/or provide an indication of any of the data sensed and/or produced by the server 228 and/or any other components depicted in FIGS. 1 and 2.

The communication component 236 may be configured to communicate (i.e., send and/or receive signals) with the electronic device 202, the electronic device 248 and/or other devices included in FIGS. 1 and 2. The communication component 236 may include, for example, circuits, program components, and one or more transmitters and/or receivers for communicating wirelessly with one or more other devices such as, for example, the electronic device 202 and/or the electronic device 248. According to various embodiments, the communication component 236 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 236 may include any combination of hardware, software, and/or firmware configured to facilitate establishing, maintaining, and using any number of communication links.

The power source 238 provides electrical power to the other operative components (e.g., the processor 230, the memory 232, the I/O component 234, and/or the communication component 236, and may be any type of power source suitable for providing the desired performance and/or longevity requirements of the server 228. In various embodiments, the power source 238 may include one or more batteries, which may be rechargeable (e.g., using an external energy source). The power source 238 may include one or more capacitors, energy conversion mechanisms, and/or the like.

In embodiments, the electronic device 248 may be accessible by a clinician for review and/or analysis of a representation of one or more of the characteristics 218A, 220A, 222A, a representation of the subject's cardiac health, a representation of a trend of the subject's cardiac health, and/or a representation of the risk associated with the subject's cardiac health. In response, the clinician may communicate to the electronic device 202 one or more diagnoses, courses of treatment, lifestyle changes, and/or the like.

The processor 252 may include, for example, one or more processing units, one or more pulse generators, one or more controllers, one or more microcontrollers, and/or the like. The processor 252 may be any arrangement of electronic circuits, electronic components, processors, program components and/or the like configured to store and/or execute programming instructions, to direct the operation of the other functional components of the electronic device 248 and may be implemented, for example, in the form of any combination of hardware, software, and/or firmware.

In embodiments, the processor 252 may be, include, or be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more central processing units (CPUs), software, hardware, firmware, or any combination of these and/or other components. According to embodiments, the processor 252 may include a processing unit configured to communicate with memory 250 to execute computer-executable instructions stored in the memory 250. As indicated above, although the processor 252 is referred to herein in the singular, the processor 252 may be implemented in multiple instances, distributed across multiple sensing devices, instantiated within multiple virtual machines, and/or the like.

In embodiments, the memory 250 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory stores computer-executable instructions for causing the processor to implement aspects of embodiments of system components discussed herein and/or to perform aspects of embodiments of methods and procedures discussed herein.

Computer-executable instructions stored on memory 250 may include, for example, computer code, machine-useable instructions, and the like such as, for example, program components capable of being executed by one or more processors associated with the computing device. Program components may be programmed using any number of different programming environments, including various languages, development kits, frameworks, and/or the like. Some or all of the functionality contemplated herein may also, or alternatively, be implemented in hardware and/or firmware.

In embodiments, the I/O component 254 may include a user interface configured to present information to a user or receive indication from a user. For example, the I/O component 254 may include and/or be coupled to a display device, a printing device, a speaker, a light emitting diode (LED), and/or the like, and/or an input component such as, for example, a button, a joystick, a microphone, a satellite dish, a scanner, a printer, a wireless device, a keyboard, a pen, a voice input device, a touch input device, a touch-screen device, an interactive display device, a mouse, and/or the like. In embodiments, the I/O component 254 may be used to present and/or provide an indication of any of the data sensed and/or produced by the electronic device 248 and/or any other components depicted in FIGS. 1 and 2.

The communication component 258 may be configured to communicate (i.e., send and/or receive signals) with the electronic device 202, the server 228 and/or other devices included in FIGS. 1 and 2. The communication component 258 may include, for example, circuits, program components, and one or more transmitters and/or receivers for communicating wirelessly with one or more other devices such as, for example, the electronic device 202 and/or the server 228. According to various embodiments, the communication component 258 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 258 may include any combination of hardware, software, and/or firmware configured to facilitate establishing, maintaining, and using any number of communication links.

The power source 260 provides electrical power to the other operative components (e.g., the processor 252, the memory 250, the I/O component 254, and/or the communication component 258, and may be any type of power source suitable for providing the desired performance and/or longevity requirements of the electronic device 248. In various embodiments, the power source 260 may include one or more batteries, which may be rechargeable (e.g., using an external energy source). The power source 260 may include one or more capacitors, energy conversion mechanisms, and/or the like.

The illustrated embodiment shown in FIG. 2 is not intended to suggest any limitation as to the scope of use or functionality of embodiments of the present disclosure. The illustrative embodiment should not be interpreted as having any dependency or requirement related to any single component or combination of components illustrated therein. Additionally, various components depicted in FIG. 2 may be, in embodiments, integrated with various ones of the other components depicted therein (and/or components not illustrated), all of which are considered to be within the ambit of the subject matter disclosed herein.

FIG. 3 is a graph 300 depicting a characteristic of a subject, in accordance with embodiments of the present disclosure. The graph illustrates how a characteristic of a voice sample can be compared against a characteristic of a baseline voice sample to determine the cardiac health of a subject.

The illustrated graph 300 includes characteristic 302 (e.g., a characteristics of one or more of the characteristics 218A, 220A, 222A) as a function of a parameter 304. Example characteristics include but are not limited to the characteristics 218A, 220A, 222A discussed in relation to FIG. 2. Further, the graph 300 includes a characteristic of a baseline voice sample 306. In embodiments, the baseline voice sample may be the same or similar as the baseline voice sample discussed in relation to the other FIGs. For example, the baseline voice sample may be received from the subject. As another example, the baseline voice sample may be received from a group of subjects that includes or doesn't include the subject for which the cardiac health is being determined. In embodiments the group of subjects may have at least one statistical characteristic that is similar to a statistical characteristic of the subject for which the cardiac health is being determined.

The graph 300 also includes a characteristic of a first voice sample 308, a boundary condition for the characteristic 310, and a characteristic of a second voice sample 312. In the illustrated example, the characteristic of the first voice sample 308 is located closer to the characteristic of the baseline voice sample 306 than the boundary condition for the characteristic 310. This may indicate that the cardiac health of the subject is within an acceptable range. Conversely, the characteristic of the second voice sample 312 is located farther away from the characteristic of the baseline voice sample 306 than the boundary condition for the characteristic 310. This may indicate that the cardiac health of the subject is not within an acceptable range and, therefore, may indicate the subject has one or more cardiac health related problems. In embodiments, the characteristic 302 may be determined at a plurality of times.

FIG. 4 is a graph 400 depicting a trend of a subject's cardiac health, in accordance with embodiments of the present disclosure. As illustrated, the graph 400 includes the subject's cardiac health at a plurality of times. Specifically, the graph includes the subjects' cardiac health at a first time 402, second time 404, third time 406, fourth time 408, and fifth time 410. By tracking the subject's cardiac health at a plurality of times, the subject and/or a clinician can determine whether the subject's cardiac health is getting better, worse or is static. In embodiments, a clinician may also prescribe one or more lifestyle changes, one or more surgical procedures, one or more medications, and/or the like based on the trend of the subject's cardiac health. Additionally or alternatively, a clinician may determine the effectiveness of one or more lifestyle changes, one or more surgical procedures, one or more medication(s), and/or the like based on the trend of the subject's cardiac health.

FIG. 5 is a graph 500 depicting a risk stratification of a subject's cardiac health, in accordance with embodiments of the present disclosure. As illustrated, the graph 500 depicts a low risk category 502, a medium risk category 504, and a high risk category 506. Further, the graph depicts the subject's cardiac health 508, which is above the low risk category 502, but below the medium risk category 504. The graph 500 also depicts the subject's cardiac health trend 510, which is above the medium risk category 504, but below the high risk category 506, indicating the risk associated with the subject's cardiac health has been increasing and in the future the subject's cardiac health will likely progress to between the medium risk category 504 and the high risk category 506. As a result, the subject and/or the clinician may develop a plan to slow and/or reverse the subject's cardiac health trend.

FIG. 6 is a flow diagram of a method 600 for determining the cardiac health of a subject using voice analysis, in accordance with embodiments of the present disclosure. In embodiments, the method 600 comprises prompting a subject for a voice sample (block 602). In embodiments, the subject may be prompted for a voice sample according to any of the embodiments discussed in relation to the other FIGs. In embodiments, the method 600 further comprises receiving a voice sample from a subject (block 604). In embodiments, the method 600 also comprises receiving sensed data (from, e.g., a sensor 108) and/or health data (block 606). In embodiments, the sensed data and/or the health data may be the same or similar as the sensed data 214 and/or the health data 240, respectively, discussed in relation to the other FIGs. In embodiments, the method 400 further comprises storing a baseline voice sample (block 608). In embodiments, the baseline voice sample may be the same or similar as the baseline voice sample discussed in relation to the other FIGs. For example, the baseline voice sample may be received from the subject. Additionally or alternatively, the baseline voice sample may be received from the subject at a first time, wherein the voice sample is received from the subject at a second time such that the second time is after the first time. As another example, the baseline voice sample may be received from a group of subjects that includes or doesn't include the subject for which the cardiac health is being determined. In embodiments the group of subjects may have at least one statistical characteristic that is similar to a statistical characteristic of the subject for which the cardiac health is being determined.

In embodiments, the method 600 comprises determining one or more characteristics of the voice sample (block 610). In embodiments, the one or more characteristics may be the same or similar as the one or more characteristics 218A, 220A discussed in relation to the other FIGs. In embodiments, one or more characteristics may be determined for voice sample received from the group of subjects and may be the same or similar as the characteristics 222A discussed in relation to the other FIGs. For example, the one or more characteristics may be a frequency distribution of the voice sample.

In embodiments, the method 600 may further comprise determining the subject's cardiac health based on the one or more characteristics (block 612). In embodiments, determining the subject's cardiac health may be determined in the same or a similar manner as determining the subject's cardiac health described in relation to the other FIGs. For example, the subject's cardiac health may be determined using machine learning techniques. Additionally or alternatively, the subject's cardiac health may be determined by comparing the one or more characteristics of the subject's voice sample to one or more characteristics from a baseline voice sample.

In embodiments, the method 600 comprises stratifying the subject's cardiac health (block 614). In embodiments, the subject's cardiac health may be stratified in a same or similar manner as the embodiments described in relation to the other FIGs. In embodiments, the method 600 comprises determining a trend of the subject's cardiac health (block 616). In embodiments, determining a trend of the subject's cardiac health may be performed in a same or similar manner as the embodiments described in relation to the other FIGs. For example, the subject's cardiac health determined at a first time may be determined in comparison to the subject's cardiac health at a second time (and a third time, fourth time, etc.) In embodiments, the method 600 comprises outputting to a display device a representation of the subject's cardiac health, the trend, and/or the risk stratification (block 618). In embodiments, outputting to a display device a representation of the subject's cardiac health, the trend, and/or the risk stratification may be the same or similar to the embodiments depicted in relation to the other FIGs.

The illustrative method 600 shown in FIG. 6 is not intended to suggest any limitation as to the scope of use or functionality of embodiments of the present disclosure. Neither should the illustrative method 600 be interpreted as having any dependency or requirement related to any single step or combination of steps illustrated therein. Additionally, various steps depicted in FIG. 6 may be, in embodiments, integrated with various ones of the other steps depicted therein (and/or steps not illustrated), all of which are considered to be within the ambit of the present disclosure.

As set forth above, due to the embodiments described herein, intervention to increase a subject's cardiac health may be taken prior to the subject having to visit an emergency room, which may save money and/or resources spent by or on the subject.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A method for tracking cardiac health of a subject using voice analysis, the method comprising:
receiving a voice sample from the subject;
determining one or more characteristics of the voice sample; and
determining the subject's cardiac health based on the one or more characteristics.

2. The method of claim 1, wherein determining the subject's cardiac health comprises determining the subject's cardiac health using machine learning techniques.

3. The method of claim 1, further comprising storing a baseline voice sample and wherein determining the subject's cardiac health comprises comparing the one or more characteristics of the voice sample to one or more characteristics of the baseline voice sample.

4. The method of claim 3, wherein the baseline voice sample is received from the subject.

5. The method of claim 3, wherein the baseline voice sample is received from a group of individuals, wherein each individual of the group of individuals has at least one statistical characteristic that is similar to a statistical characteristic of the subject.

6. The method of claim 1, wherein determining one or more characteristics of the voice sample comprises determining a frequency distribution of the voice sample and wherein determining the subject's cardiac health comprises determining the subject's cardiac health based on the frequency distribution of the voice sample.

7. The method of claim 1, further comprising determining a cardiac health trend for the subject based on the subject's cardiac health determined at a first time and a second time, the second time occurring after the first time.

8. The method of claim 1, further comprising stratifying the subject into a risk category based on the subject's cardiac health.

9. The method of claim 1, further comprising receiving sensed data from a sensor associated with the subject and wherein determining the subject's cardiac health is based on the sensed data.

10. The method of claim 1, further comprising receiving health data associated with the subject and wherein determining the subject's cardiac health comprises determining the subject's cardiac health based on the health data.

11. The method of claim 1, wherein determining the subject's cardiac health comprises receiving whether the subject has experienced or is experiencing preserved ejection fraction or reduced ejection fraction and wherein determining the subject's cardiac health comprises determining the subject's cardiac health based on the whether the subject has experienced or is experiencing preserved ejection fraction or reduced ejection fraction.

12. The method of claim 1, wherein receiving a voice sample from the subject comprises receiving a voice sample from the subject during a voice call in which the subject is participating.

13. The method of claim 1, further comprising prompting the subject to elicit the voice sample.

14. The method of claim 1, further comprising outputting to a display device a representation of the subject's cardiac health.

15. A non-transitory computer readable medium having a computer program stored thereon for determining cardiac health of a subject using voice analysis, the computer program comprising instructions for causing one or more processors to:
receive a voice sample from the subject;
determine one or more characteristics of the voice sample; and
determine the subject's cardiac health based on the one or more characteristics.

16. The non-transitory computer readable medium of claim 15, wherein to determine the subject's cardiac health, the computer program comprises instructions to determine the subject's cardiac health using machine learning techniques.

17. The non-transitory computer readable medium of claim 15, the computer program comprising instructions to store a baseline voice sample and wherein to determine the subject's cardiac health, the computer program comprises instructions to compare the one or more characteristics of the voice sample to one or more characteristics of the baseline voice sample.

18. The non-transitory computer readable medium of claim 17, wherein the baseline voice sample is received from the subject and/or a group of individuals, wherein each individual of the group of individuals has at least one statistical characteristic that is similar to a statistical characteristic of the subject.

19. The non-transitory computer readable medium of claim 15, the computer program comprising instructions to determine a cardiac health trend for the subject based on the subject's cardiac health determined at a first time and a second time, the second time occurring after the first time.

20. The non-transitory computer readable medium of claim 15, the computer program comprising instructions to stratify the subject into a risk category based on the subject's cardiac health.
